(19) United States
(12) Patent Application Publication
(10) Pub. No.: US 2021/0393247 A1
(43) Pub. Date: Dec. 23, 2021

(54) METHODS FOR CARRYING OUT A CARDIAC PROCEDURE

(71) Applicant: Baylis Medical Company Inc., Montreal (CA)

(72) Inventors: Eduardo Moriyama, Richmond (CA); Kaylie Lau, Toronto (CA); Charlene Leung, Scarborough (CA)

(21) Appl. No.: 17/345,056

(22) Filed: Jun. 11, 2021

Related U.S. Application Data
(60) Provisional application No. 63/040,042, filed on Jun. 17, 2020.

(51) Int. Cl. A61B 17/00; A61B 17/34

(52) U.S. Cl. CPC A61B 17/00234; A61B 17/3423; A61B 2017/00336; A61B 2017/00247; A61B 2017/3425; A61B 17/3468

(57) ABSTRACT

A method for carrying out a cardiac procedure includes: a. via an inferior artery, advancing a perforating tip of a perforation device towards an aorta; b. positioning the perforating tip adjacent a wall of the aorta, proximate a left pulmonary artery; and c. advancing the perforating tip to perforate through the wall of the aorta and then through a wall of the left pulmonary artery, to create a pathway between the aorta and the left pulmonary artery.

### FIELD

This document relates to medical procedures. More specifically, this document relates to methods for carrying out a cardiac procedure.

### SUMMARY

The following summary is intended to introduce the reader to various aspects of the detailed description, but not to define or delimit any invention.

Methods for carrying out a cardiac procedure are disclosed. According to some aspects, a method for carrying out a cardiac procedure includes: a. via an inferior artery, advancing a perforating tip of a perforation device towards an aorta; b. positioning the perforating tip adjacent a wall of the aorta, proximate a left pulmonary artery; and c. advancing the perforating tip to perforate through the wall of the aorta and then through a wall of the left pulmonary artery, to create a pathway between the aorta and the left pulmonary artery.

In some examples, the perforation device is a radiofrequency perforation device, the perforating tip includes a radiofrequency perforation electrode, and step c. includes delivering radiofrequency energy from the radiofrequency perforation electrode while advancing the perforating tip.

In some examples, the method further includes: d. via the inferior artery, advancing a dilator over the perforation device to the aorta; and e. after step c., advancing a dilating tip of the dilator over the perforation device and through the pathway to dilate the pathway. The dilator can be a steerable dilator.

In some examples, the method further includes: f. via the inferior artery, advancing a sheath over the dilator and the perforation device to the aorta; and g. after step e., retracting the dilator through the sheath. The sheath can be a steerable sheath.

In some examples, the method further includes: h. after step f., exchanging the perforation device for a guidewire.

In some examples, the method further includes: h. after step g., delivering a therapeutic device to the pathway via the sheath. Step h. can include positioning a shunt in the pathway or positioning a stent in the pathway.

In some examples, the method further includes: h. via the inferior artery, exchanging the sheath for a secondary sheath; and i. delivering a therapeutic device to the pathway via the secondary sheath.

In some examples, the inferior artery is a femoral artery.

In some examples, the method further includes: d. advancing a snare towards the left pulmonary artery via a venous access site; and e. after step c., snaring the perforation device with the snare. Step d. can include advancing the snare towards the left pulmonary artery via a femoral vein, a hepatic vein, or a superior vein. In some examples, the method further includes: f. after step e., retracting the snare to advance the perforating tip out of the body towards the venous access site.

In some examples, the method further includes delivering a therapeutic device over the perforation device towards the pathway, via the venous access site.

In some examples, at least one of fluoroscopy, angiography, electro-anatomical mapping, intracardiac echocardiography, and transesophageal echocardiography is carried out concurrently with at least one of steps a. to c.

In some examples, the method further includes confirming the creation of the pathway with at least one of fluoroscopy, electro-anatomical mapping, pressure measurement, contrast injection, and echocardiography.

In some examples, the method further includes advancing a balloon catheter over the perforation device to dilate the pathway.

In some examples, the method further includes using an anchor system to bring the aorta and the left pulmonary artery together.

In some examples, the perforation device is a mechanical perforation device.

### BRIEF DESCRIPTION OF THE DRAWINGS

The accompanying drawings are for illustrating examples of articles, methods, and apparatuses of the present disclosure and are not intended to be limiting. In the drawings:

FIG. 1 is a perspective view of an example perforation system;

FIG. 2 is a schematic view showing a first step of a method for carrying out a cardiac procedure;

FIG. 3 is another schematic view showing the first step of the method of FIG. 1;

FIG. 4 is a schematic view showing a step subsequent to that of FIGS. 2 and 3;

FIG. 5 is a schematic view showing a step subsequent to that of FIG. 4;

FIG. 6 is a schematic view showing a step subsequent to that of FIG. 5;

FIG. 7 is a schematic view showing a step subsequent to that of FIG. 6;

FIG. 8 is a schematic view showing a step subsequent to that of FIG. 7;

FIG. 9 is a schematic view showing a step subsequent to that of FIG. 8;

FIG. 10 is a schematic view showing a step subsequent to that of FIG. 9;

FIG. 11 is a schematic view showing a step subsequent to that of FIG. 10;

FIG. 12 is a schematic view showing a step subsequent to that of FIG. 13; and

FIG. 13 is a schematic view showing a step subsequent to that of FIG. 12.

### DETAILED DESCRIPTION

Various apparatuses or processes or compositions will be described below to provide an example of an embodiment of the claimed subject matter. No example described below limits any claim and any claim may cover processes or apparatuses or compositions that differ from those described below. The claims are not limited to apparatuses or processes or compositions having all of the features of any one apparatus or process or composition described below or to features common to multiple or all of the apparatuses or processes or compositions described below. It is possible that an apparatus or process or composition described below is not an embodiment of any exclusive right granted by issuance of this patent application. Any subject matter described below and for which an exclusive right is not granted by issuance of this patent application may be the subject matter of another protective instrument, for example, a continuing patent application, and the applicants, inventors or owners do not intend to abandon, disclaim or dedicate to the public any such subject matter by its disclosure in this document.

Generally disclosed herein are methods for carrying out cardiac procedures, and more specifically, cardiac procedures in which a pathway (also referred to as a “communication”) is created between the aorta (e.g. the descending aorta) and the left pulmonary artery of a patient. Such procedures can be carried out, for example, to allow for the insertion of a therapeutic device (e.g. a shunt or a stent) into the pathway, to treat idiopathic pulmonary arterial hypertension or other heart defects.

The methods disclosed herein involve the creation of a pathway between the aorta and the left atrium via an inferior approach—that is, the aorta can be approached via an inferior artery (e.g. the femoral artery), and a perforation can be created in the wall of the aorta and then into the wall of the left atrium

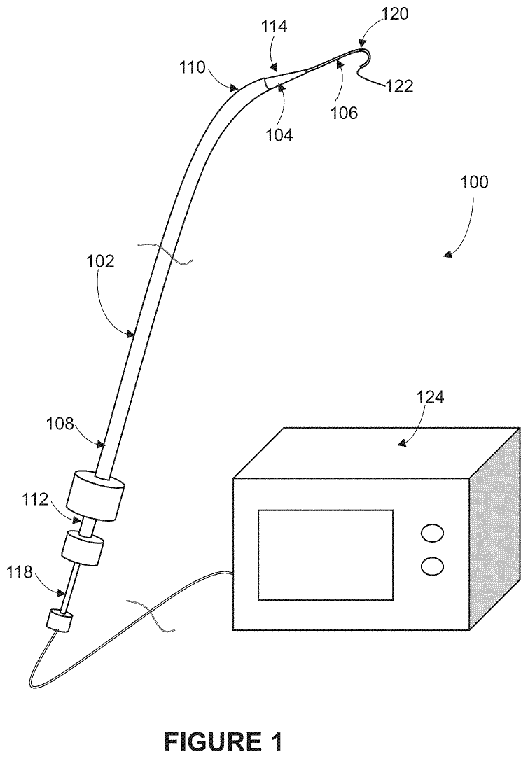

Referring first to FIG. 1, a system 100 for carrying out a cardiac procedure is shown. In the example shown, the system 100 is a perforation system, and includes a sheath 102, a dilator 104 (the majority of which is within the sheath 102 in FIG. 1), and a perforation device 106 (the majority of which is within the dilator 104 in FIG. 1).

The sheath 102 can be used to guide various other devices (e.g. the dilator 104, or therapeutic devices such as a stent or shunt) towards a target location in a patient's body (e.g. the aorta). The sheath 102 has a proximal portion 108 and a distal portion 110, and a lumen (not shown) extends through the sheath 102 from the proximal portion 108 to the distal portion 110. The sheath 102 can optionally have a fixed curve, or can be steerable (i.e. the curve can be changed, optionally in more than one plane).

The dilator 104 can be used to dilate a perforation, and has a proximal portion 112 and a distal portion 114 having a dilating tip. A lumen (not shown) extends through the dilator from the proximal portion 112 to the distal portion 114. The dilator 104 can optionally have a fixed curve, or can be steerable (i.e. the curve can be changed, optionally in more than one plane). The dilator 104 can optionally be flexible, to allow it to be compatible with a steerable sheath.

The perforation device 106 can be used to perforate a target anatomical structure (e.g. a wall of the aorta), and has a proximal portion 118 and a distal portion 120. The distal portion 120 has a perforating tip 122, and in the example shown is biased towards a J-shape, to prevent inadvertent perforation of anatomical structures with the perforating tip 122. In the example shown, the perforation device 106 is a radiofrequency (RF) perforation device, and the perforating tip 122 includes a radiofrequency perforation electrode. The system 100 further includes a radiofrequency generator 124, which can be connected to the perforation device 106 to deliver RF energy to the perforation electrode 122, and to one or more grounding pads (not shown). The perforation device 106 can also serve as a support guidewire.

In alternative examples, the perforation device can be a mechanical perforation device, and the perforating tip can include a sharp point.

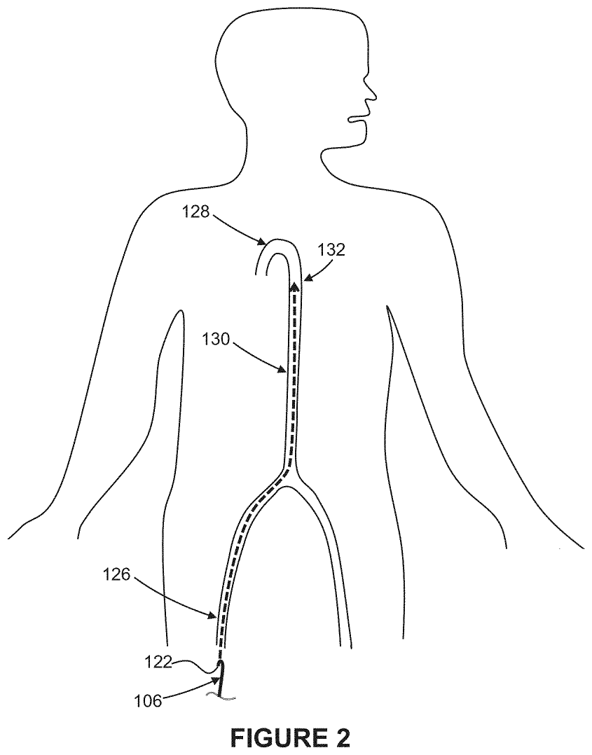

Referring now to FIGS. 2 to 13, a method for carrying out a cardiac procedure, and specifically for creating a pathway between an aorta and a left pulmonary artery via an inferior artery, will be described. The method will be described with regard to the system 100 shown in FIG. 1; however, the method is not limited to the system 100.

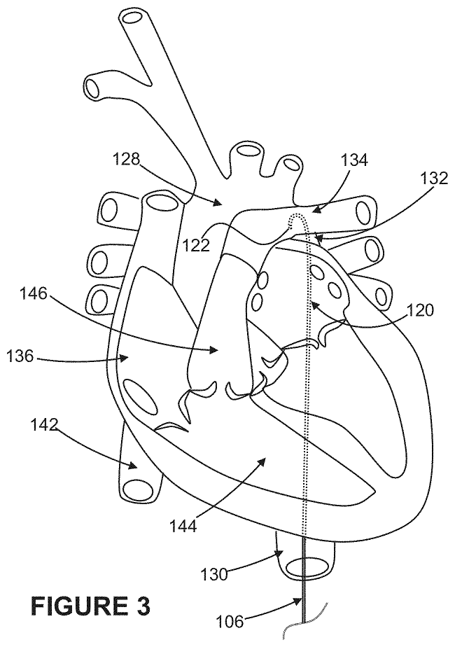

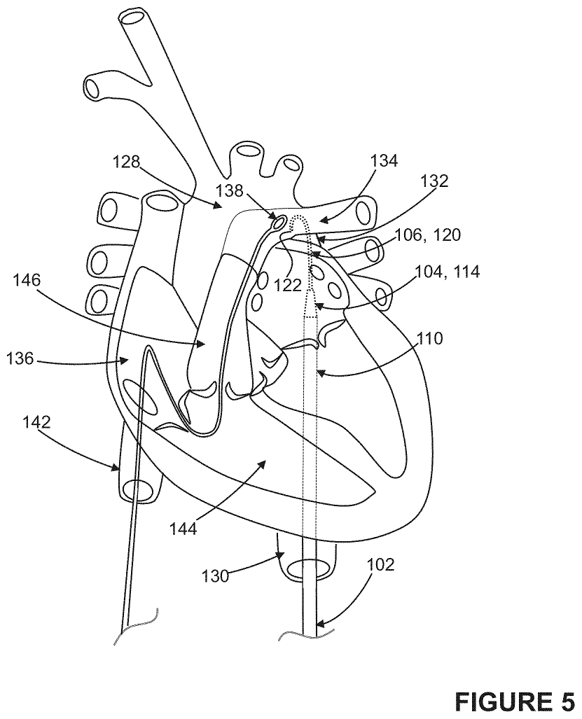

Referring to FIG. 2, as a first step, an inferior artery (which in the example shown is a femoral artery 126) can be percutaneously accessed (e.g. using a procedure such as a Seldinger technique) and the perforating tip 122 of the perforation device 106 can be advanced into the inferior artery and towards the aorta 128. Referring also to FIG. 3, in the example shown, the perforating tip 122 is advanced via the femoral artery 126 towards the abdominal aorta 130 and then into the descending aorta 132. In FIG. 3 (and also in FIG. 5), features that are within the descending aorta 132 (e.g. the distal portion 120 of the perforation device 106) are shown in dotted line, as much of the descending aorta 132 is behind the heart and the left pulmonary artery 134.

Optionally, during advancement of the perforating tip 122, the position of the perforating tip 122 can be confirmed using fluoroscopy (e.g. in examples wherein the perforation device 106 includes one or more radiopaque markers or features), angiography, electro-anatomical mapping (EAM) (e.g. to confirm real-time positioning of the perforating tip 122 using real-time or pre-determined computerized tomography data, in conjunction with a catheter or guidewire with one or more EAM markers in the right atrium 136), intracardiac and/or transesophageal echocardiography (ICE and/or TEE) (e.g. using echogenic markers or features on the perforation device 106).

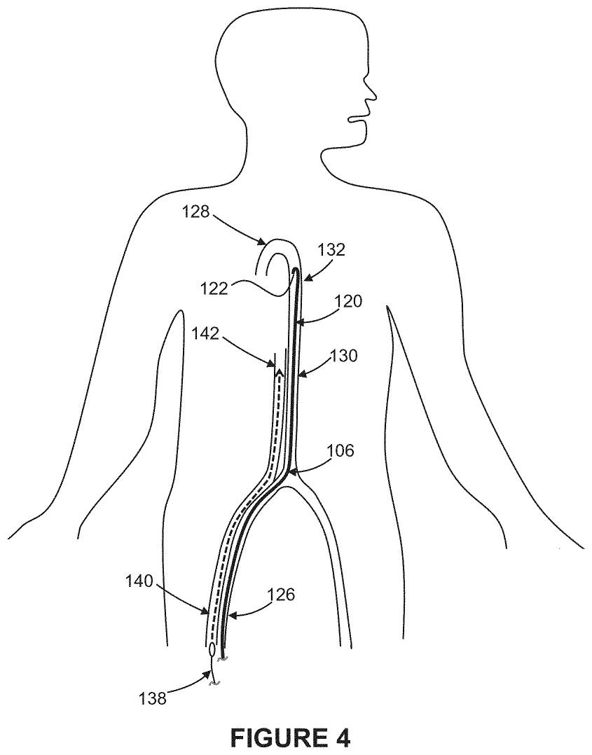

Referring to FIGS. 3 and 4, as a next step (or earlier or later in the method, for example prior to the previous steps), a snare 138 can be advanced percutaneously towards the left pulmonary artery 134. The snare 138 can be advanced, for example, via a venous access site such as the femoral vein, the hepatic vein, or a superior vein. In the example shown, the snare 138 is advanced via the femoral vein 140, the inferior vena cava 142, the right atrium 136, the right ventricle 144, and the pulmonary trunk 146, until the snare 138 reaches the left pulmonary artery 134, as shown in FIG. 5.

Referring still to FIG. 5, as a next step, the dilator 104 and the sheath 102 may be advanced towards the descending aorta 132 via the femoral artery 126 (not shown in FIG. 5) and the abdominal aorta 130. The dilator 104 and sheath 102 can be advanced together over the perforation device 106, with the dilator 104 received in the sheath 102, or can be advanced in sequence, e.g. by advancing the dilator 104 over the perforation device 106 and then advancing the sheath 102 over the dilator 104. The dilator 104 and sheath 102 can be advanced until the dilating tip of the dilator 104 is flush with the perforating tip 122 of the perforation device 106. In alternative examples, a large steerable dilator (not shown) can replace the sheath 102 and dilator 104.

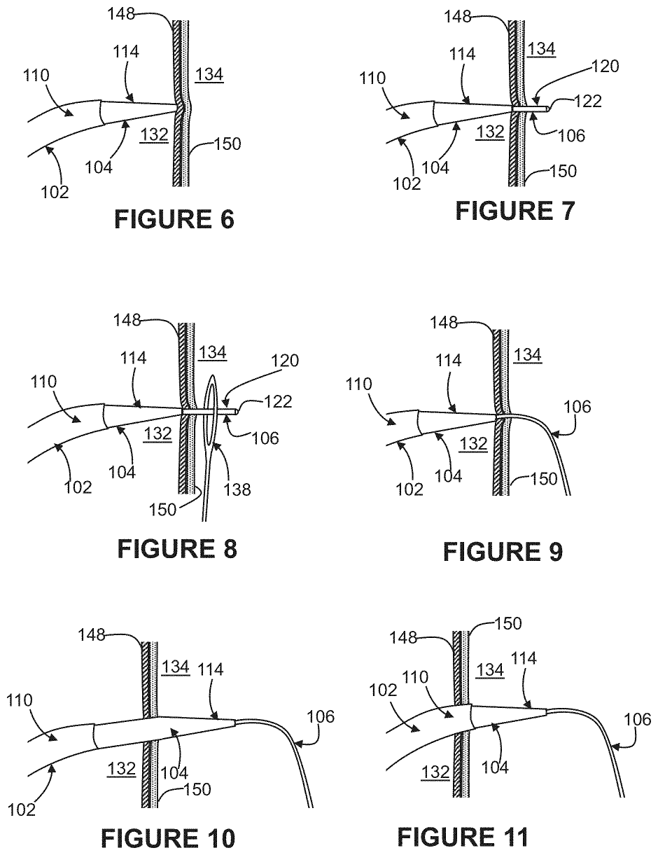

With the distal portion 110 of the sheath 102, distal portion 114 of the dilator 104, and distal portion 120 of the perforation device 106 in the descending aorta 132, the sheath 102 and dilator 104 can be maneuvered to direct the perforating tip 122 towards a desired perforation site—i.e. the wall 148 of the descending aorta 132, proximate a wall 150 of the left pulmonary artery 134—as shown in FIG. 6. This can be achieved by steering the sheath 102 and/or dilator 104 (in examples wherein the sheath 102 and/or the dilator 104 are steerable), or by adjusting the position of the sheath 102 and/or dilator 104. This step can optionally be facilitated using fluoroscopy (e.g. in examples wherein the perforation device 106 includes one or more radiopaque markers or features), angiography, electro-anatomical mapping (EAM) (e.g. to confirm real-time positioning of the perforating tip 122 using real-time or pre-determined computerized tomography data, in conjunction with a catheter or guidewire with one or more EAM markers in the right atrium 136, intracardiac and/or transesophageal echocardiography (ICE and/or TEE) (e.g. using echogenic markers or features on the perforation device 106).

Referring to FIG. 7, the perforating tip 122 can then be advanced to perforate through the wall 148 of the descending aorta 132 and through the wall 150 of the left pulmonary artery 134, to create a pathway between the descending aorta 132 and the left pulmonary artery 134. More specifically, the radiofrequency electrode can be energized with radiofrequency energy, and the perforation device 106 can be advanced to perforate through the wall 148 of the descending aorta 132 and through the wall 150 of the left pulmonary artery 134. Optionally, creation of the pathway can be confirmed using fluoroscopy, electro-anatomical mapping, pressure measurement, contrast injection, and intracardiac and/or transesophageal echocardiography.

Referring to FIGS. 8 and 9, the snare 138 can then be used to capture the distal portion 120 of the perforation device 106, and the snare 138 can then be retracted to advance the perforation device 106 towards the venous access site (e.g. the femoral vein 140, not visible in FIGS. 8 and 9), optionally to externalize the perforating tip 122. Optionally, the perforation device 106 can be “flossed” to enlarge the pathway.

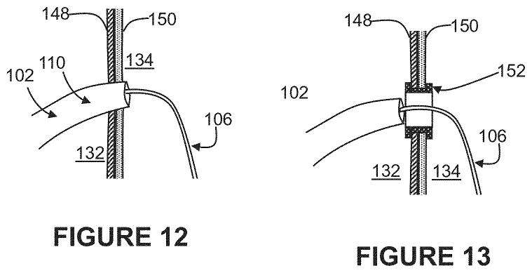

Referring to FIG. 10, the dilating tip of the dilator 104 can then be advanced over the perforation device 106 and through the pathway, to dilate the pathway. Referring to FIG. 11, the sheath 102 can then be advanced over the dilator 104 and through the pathway. Referring to FIG. 12, the dilator 104 (not visible in FIG. 12) can then be retracted back towards the femoral artery 126 (not visible in FIG. 12) and removed from the body, leaving the sheath 102 and the perforation device in place 106 in the pathway.

Optionally, after perforation, the perforation device 106 can be exchanged for another wire (either via the arterial access site or the venous access site), such as a relatively stiff guidewire.

Optionally, after perforation, an anchor device such as a balloon can be advanced via the sheath 102 and used to bring the descending aorta 132 and the left pulmonary artery 134 together.

Optionally, after perforation, a balloon catheter can be advanced over the perforation device 106 and via the sheath 102 to enlarge the pathway.

Referring to FIG. 13, with the sheath 102 and the perforation device 106 (or another guidewire) in place in the pathway, a therapeutic device can be delivered to the pathway via the sheath 102, over the perforation device 106. The therapeutic device can be for, example, a shunt (e.g. shunt 152, shown in FIG. 13) or a stent that is positioned in the pathway.

Alternatively, the sheath 102 can be retracted towards the femoral artery 126, and a secondary sheath—e.g. a large bore sheath designed for therapeutic device delivery—can be advanced over the perforation device 106 (or another guidewire) via the femoral artery 126. The secondary sheath can then be used to deliver a therapeutic device to the pathway.

Alternatively, delivery of the therapeutic device can be achieved by delivering the therapeutic device over the perforation device 106 via the venous access site (e.g. the femoral vein 140). For example, a secondary sheath (e.g. a large bore sheath, not shown) can be advanced over the perforation device 106 via the venous access site, to the left pulmonary artery 134. The secondary sheath can then be used to deliver the therapeutic device.

While the above description provides examples of one or more processes or apparatuses or compositions, it will be appreciated that other processes or apparatuses or compositions may be within the scope of the accompanying claims.

To the extent any amendments, characterizations, or other assertions previously made (in this or in any related patent applications or patents, including any parent, sibling, or child) with respect to any art, prior or otherwise, could be construed as a disclaimer of any subject matter supported by the present disclosure of this application, Applicant hereby rescinds and retracts such disclaimer. Applicant also respectfully submits that any prior art previously considered in any related patent applications or patents, including any parent, sibling, or child, may need to be re-visited.

We claim:

1. A method for carrying out a cardiac procedure, comprising:
a. via an inferior artery, advancing a perforating tip of a perforation device towards an aorta;
b. positioning the perforating tip adjacent a wall of the aorta, proximate a left pulmonary artery; and
c. advancing the perforating tip to perforate through the wall of the aorta and then through a wall of the left pulmonary artery, to create a pathway between the aorta and the left pulmonary artery.

2. The method of claim 1, wherein the perforation device is a radiofrequency perforation device, the perforating tip comprises a radiofrequency perforation electrode, and step c. comprises delivering radiofrequency energy from the radiofrequency perforation electrode while advancing the perforating tip.

3. The method of claim 1, further comprising:
d. via the inferior artery, advancing a dilator over the perforation device to the aorta;
e. after step c., advancing a dilating tip of the dilator over the perforation device and through the pathway to dilate the pathway.

4. The method of claim 3, wherein the dilator is a steerable dilator.

5. The method of claim 3, further comprising:
f. via the inferior artery, advancing a sheath over the dilator and the perforation device to the aorta;
g. after step e., retracting the dilator through the sheath.

6. The method of claim 5, further comprising:
h. after step f., exchanging the perforation device for a guidewire.

7. The method of claim 5, further comprising:
h. after step g., delivering a therapeutic device to the pathway via the sheath.

8. The method of claim 7, wherein step h. comprises positioning a shunt in the pathway.

9. The method of claim 7, wherein step h. comprises positioning a stent in the pathway.

10. The method of claim 5, further comprising:
h. via the inferior artery, exchanging the sheath for a secondary sheath; and
i. delivering a therapeutic device to the pathway via the secondary sheath.

11. The method of claim 5, wherein the sheath is a steerable sheath.

12. The method of claim 1, wherein the inferior artery is a femoral artery.

13. The method of claim 1, further comprising:
d. advancing a snare towards the left pulmonary artery via a venous access site; and
e. after step c., snaring the perforation device with the snare.

14. The method of claim 13, wherein step d. comprises advancing the snare towards the left pulmonary artery via a femoral vein, a hepatic vein, or a superior vein.

15. The method of claim 13, further comprising:
f. after step e., retracting the snare to advance the perforating tip out of the body towards the venous access site.

16. The method of claim 15, further comprising delivering a therapeutic device over the perforation device towards the pathway, via the venous access site.

17. The method of claim 1, wherein at least one of fluoroscopy, angiography, electro-anatomical mapping, intracardiac echocardiography, and transesophageal echocardiography is carried out concurrently with at least one of steps a. to c.

18. The method of claim 1, further comprising confirming the creation of the pathway with at least one of fluoroscopy, electro-anatomical mapping, pressure measurement, contrast injection, and echocardiography.

19. The method of claim 1, further comprising advancing a balloon catheter over the perforation device to dilate the pathway.

20. The method of claim 1, further comprising using an anchor system to bring the aorta and the left pulmonary artery together.
